(19) United States
(12) Patent Application Publication
(10) Pub. No.: US 2017/0105620 A1
(43) Pub. Date: Apr. 20, 2017

(54) STEREO-OPTIC SURGICAL CONTACT LENS

(71) Applicant: Novartis AG, Basel (CH)

(72) Inventor: Steven T. Charles, Memphis, TN (US)

(21) Appl. No.: 14/886,413

(22) Filed: Oct. 19, 2015

(51) Int. Cl. A61B 3/13; G02B 13/00; G02B 21/22; A61B 3/125

(52) U.S. Cl. CPC A61B 3/132; A61B 3/125; G02B 13/0095; G02B 21/22

(57) ABSTRACT

A pair of objective lenses is integrated into a top surface of a surgical contact lens. The pair of objective lenses replaces the conventional single, shared objective lens that is typically positioned 175 mm from the ocular surface. In one example, a surgical contact lens for providing a stereo-optic view inside a patient's eye includes a first side adapted to engage the patient's eye and a second side facing away from the patient's eye. The second side includes a first optical component arranged to focus light from a first position and a second optical component arranged to focus light from a second position that is different from the first position. The first optical component is substantially coplanar with the second optical component.

### TECHNICAL FIELD

The present disclosure is directed to methods and systems for ophthalmic medical procedures, and more particularly, to methods and systems involving viewing a surgical site through a microscope system.

### BACKGROUND

Microscopes are regularly used to view the interior of a patient's eye during ophthalmic surgical operations. Typical surgical microscopes used in various surgical procedures include an eyepiece for the viewer's left eye and a separate eyepiece for the viewer's right eye. Such microscopes are referred to as binocular microscopes. Each eyepiece may be associated with an optical system that includes a series of optical components such as lenses that magnify and/or redirect light to provide a magnified image of a surgical site. In conventional binocular surgical operating microscopes, both the left eyepiece and the right eyepiece use the same, shared objective lens, which is typically placed about 120-200 millimeters from the patient's eye. The objective lens, in combination with the optical systems associated with the eyepieces, provides further magnification of the object at which the microscope is directed. In some examples, the objective lens may include a system of lenses.

In some cases, a surgical contact lens may be used along with a surgical operating microscope. A surgical contact lens typically makes direct contact with the patient's eye. It may include a series of lenses that are arranged to affect light to provide the desired view of the surgical site. For example, the surgical contact lens may be configured to neutralize the corneal optical power. The surgical contact lens may also be used to provide various viewing angles.

While microscopes may provide a magnified view of the patient's eye to a user, some features within the patient's eye may be difficult to discern, even with the help of a microscope. For example, the Internal Limiting Membrane (ILM) is approximately three microns thick, colorless, transparent, and featureless. Thus, the ILM can be difficult to view through the microscope. There is a need for continued improvement in the use and operability of microscope systems used to view the intricate features of a patient's eye during surgical operations.

### SUMMARY

According to some implementations, a pair of objective lenses is integrated into a top surface of a surgical contact lens. The pair of objective lenses replaces the conventional single, shared objective lens that is typically positioned 175 mm from the ocular surface. This increases the angle between the surgeon's left eye view-path and right eye view-path. The greater angular disparity between the two view-paths increases the stereo depth resolution. In one example, a surgical contact lens for providing a stereo-optic view inside a patient's eye includes a first side adapted to engage the patient's eye and a second side facing away from the patient's eye. The second side includes a first optical component arranged to focus light from a first position and a second optical component arranged to focus light from a second position that is different from the first position. The first optical component is substantially coplanar with the second optical component.

According to some implementations, a microscope system for providing a stereo-optic view of a patient's eye includes a surgical contact lens having a first side adapted to engage a patient's eye and a second side facing away from the patient's eye when the first side is engaged with the patient's eye, the second side comprising a first optical component and a second optical component. The system further includes an eyepiece module having a left eyepiece and a right eyepiece. The system further includes a relay optics module having a first set of optical elements to direct light along a first light path from the first optical component to the left eyepiece and a second set of optical elements to direct light along a second light path from the second optical component to the right eyepiece.

According to some implementations, a method for viewing a patient's eye with a microscope includes placing a surgical contact lens on a patient's eye, surgical contact lens comprising a first optical component and a second optical component, the first optical component and the second optical component being substantially coplanar. The method further includes positioning an eyepiece module above the patient's eye, the eyepiece module comprising a left eyepiece and a right eyepiece. The method further includes positioning a relay optics module between the surgical contact lens and the eyepiece module, the relay optics module comprising a first set of optical elements to direct light along a first light path from the first optical component to the left eyepiece and a second set of optical elements to drive light along a second light path from the second optical component to the right eyepiece.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagram showing an illustrative surgical contact lens that is arranged to provide a stereo-optic view of a patient's eye.

FIG. 2 is a diagram showing the surgical contact lens placed on a patient's eye.

FIG. 3 is a diagram showing an illustrative microscope system arranged to provide a stereo-optic view of a patient's eye.

FIG. 4 is a diagram showing a cross-sectional view of an illustrative surgical contact lens placed within an eye ring that is secured to patient's eye.

FIGS. 5A and 5B are diagrams showing top views of surgical contact lenses that are arranged to provide a stereo optic view patient's eye.

FIG. 6 is an example flowchart showing an illustrative method of viewing a patient's eye through a surgical contact lens that is arranged to provide a stereo-optic view of the patient's eye.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

As described above, the nature of the ILM is such that it is difficult to view through a microscope. According to principles described herein, viewing of the ILM and other surgical sites within the patient's eye through a microscope is enhanced by increasing the stereopsis provided by the microscope. Stereopsis refers to depth perception of a three-dimensional object obtained by viewing that object from two separate lateral positions. For example, humans are able to perceive depth because they have two eyes positioned laterally with respect to the viewing direction.

According to one example, to provide better stereoscopy, instead of having a single, shared objective lens positioned between the surgical contact lens and the eyepieces of the microscope, the surgical contact lens itself may include two separate and distinct objective lens systems formed on the top surface thereof. The shared objective lens can be replaced with a relay optics module that directs light from one of the objective lens systems to the optical system associated with the left eyepiece and directs light from the other objective lens system to the optical system associated with the right eyepiece. Thus, because the surgical contact lens provides two separate views from two distinct positions, and is much closer to the eye than a shared object lens, a greater angular disparity between two viewpoints can be achieved. This provides hyper-stereo vision of the surgical site, thus allowing better perception of the ILM and other intricate features within the patient's eye.

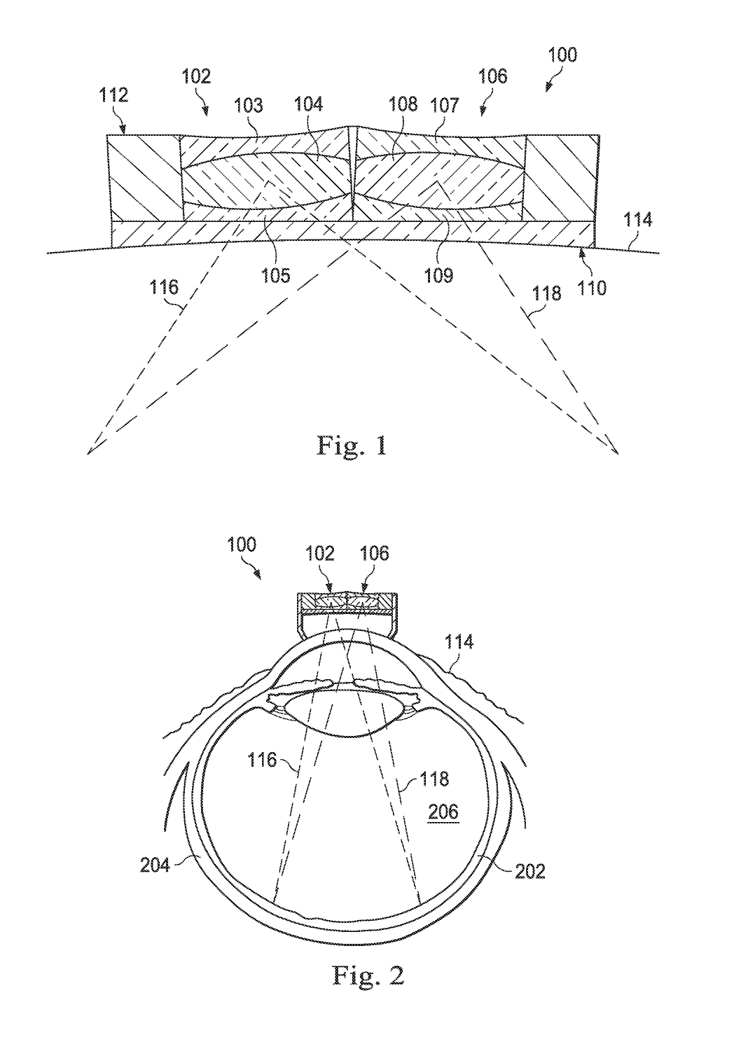

FIG. 1 is a diagram showing an illustrative surgical contact lens 100 that is arranged to provide a hyper-stereo view of a patient's eye 114. According to the present example, the surgical contact lens 100 includes a first side 110 and a second side 112. The first side 110 is arranged to directly contact the patient's eye 114. The second side 112 faces away from the patient's eye 114. The surgical contact lens 100 also includes a first optical component 102 and a second optical component 106. The first optical component 102 and the second optical component 106 act as a pair of objective lenses that replace the conventional shared objective lens. Thus, the optical components 102, 106, are adapted to serve additional functions that are not present within conventional surgical contact lenses. Additionally, the optical components 102, 106 are angled inward to increase the stereopsis provided to the viewer. The degree of angulation at which the optical components 102, 106 are positioned may vary.

In the present example, the first optical component 102 and the second optical component 106 are positioned at two separate lateral locations. Thus, each optical component 102, 106 provides a different, albeit overlapping view 116, 118. Specifically, the first optical component 102 provides a first view 116 and the second optical component 106 provides a second view 118. The first optical component 102 is positioned adjacent the second optical component 106. The first optical component 102 is substantially coplanar with the second optical component 106. As will be described in further detail below, light passing through the first optical component 102 is directed to a left eyepiece and light passing through the second optical component 106 is directed to a right eyepiece.

Both the first optical component 102 and the second optical component 106 may include a series of vertically stacked optical elements such as lenses. In the present example, optical component 102 includes a first lens 103, a second lens 104, and a third lens 105. Similarly, the second optical component 106 includes a first lens 107, a second lens 108, and a third lens 109. The characteristics of each of the lenses 103, 104, 105, 107, 108, 109 are designed to provide the desired magnification and light directing functions. Such characteristics may include, for example, the curvature, the refractive index, the thickness, and the type of lens. Various types of lenses, such as a biconcave lens, a biconvex lens, a convex-concave lens, a plano concave lens, a plano convex lens, a positive/negative meniscus lens, an aspheric lens, a converging lens, a diverging lens, and a prism lens may be used. In some examples, fewer lenses than illustrated may be used for each optical component 102, 106. In some examples, more lenses than illustrated may be used.

In some implementations, both the first optical component 102 and the second optical component 106 may each act as a separate objective lens. An objective lens is the lens that is generally the closest lens to the object under view. The objective lens is designed to focus light being reflected off the object under view and to provide magnification of that object. In some cases, a system of lenses is used as the objective lens. In other words, several objective lenses are used to provide the function of the objective lens. In the present example, lenses 103, 104, and 105 form a first objective lens system and lenses 107, 108, and 109 form a second objective lens system.

FIG. 2 is a diagram showing another view of the illustrative surgical contact lens 100 placed on a patient's eye 114. According to the present example, the optical components 102, 106 are sized and positioned to provide two distinct views 116, 118 of the interior 206 of the patient's eye 114. Specifically, the first optical component 102 provides a first view 116 and the second optical component 106 provides a second view 118. As will be explained in further detail below, the first view 116 is provided to a user's left eye and the second view 118 is provided to user's right eye. By having two separate views 116, 118, one for each of the user's eyes, the user can be provided with better depth perception of features within the interior 206 of the patient's eye 114. Here, the user may have better depth perception of the ILM 202 and the retina 204.

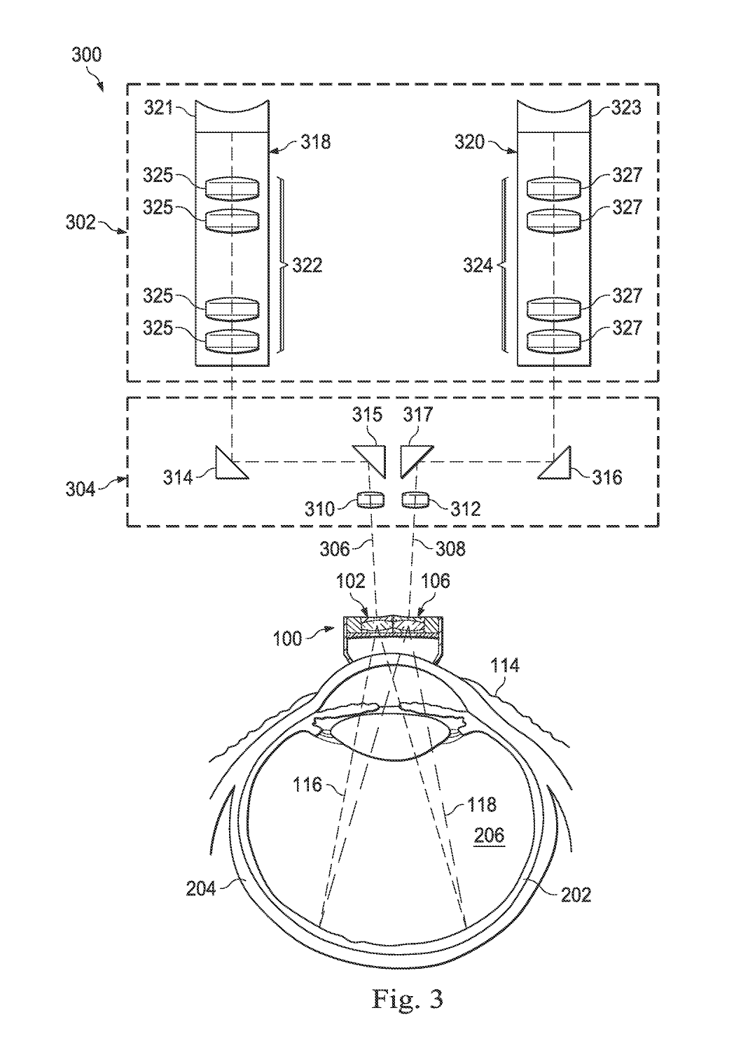

FIG. 3 is a diagram showing illustrative microscope system 300 arranged to provide a stereo-optic view of a patient's eye 114. According to the present example, the microscope system 300 includes the surgical contact lens 100, an eyepiece module 302, and a relay optics module 304. The relay optics module 304 is positioned between the surgical contact lens 100 and the eyepiece module 302.

The eyepiece module 302 includes a first optical system 318 and a second optical system 320. The first optical system 318 includes a first eyepiece 321 and a first set 322 of optical elements 325. Similarly, the second optical system 320 includes a second eyepiece 323 and a second set 324 of optical elements 327. Both the optical elements 325, 327 from the first set 322 and the second set 324 may be formed of a series of lenses that are designed to provide the desired magnification and the desired light directing properties. The optical elements 325, 327 within the first optical system 318 and the second optical system 320 may include biconcave lenses, biconvex lenses, convex-concave lenses, plano concave lenses, plano convex lenses, positive/negative meniscus lenses, aspheric lenses, converging lenses, diverging lenses, prism lenses, and other lenses. Additionally, the optical elements 325, 327 may include focusing lenses, zooming lenses, filters, gratings, or other optical elements.

The relay optics module 304 is designed to direct light from the surgical contact lens 100 to the eyepiece module 302. Specifically, the relay optics module 304 directs light along a first light path 306 from the first optical component 102 of the surgical contact lens 100 to the first optical system 318 of the eyepiece module 302. In the present example, the relay optics module 304 includes mirrors 314, 315, 316, 317 to direct light from the surgical contact lens 100 to the respective optical systems 318, 320. Additionally the relay optics module 304 may include optical elements such as lenses 310, 312 through which light passes. In other examples, however, there may be no optical elements between the surgical contact lens 100 and the mirrors 315, 317.

In some examples, the relay optics module 304 and the eyepiece module 302 are specifically positioned with respect to the surgical contact lens 100. The magnification provided by the microscope system 300 may be affected by distances between the lenses within the microscope system 300. Specifically, magnification is affected by the distance between the surgical contact lens 100 and the relay optics module 304. Additionally, magnification is affected by the distance between the relay optics module 304 and the eyepiece module 302. Accordingly, before surgical operations begin, the relay optics module 304 and the eyepiece module 302 are positioned as desired to give the user the desired magnification of the surgical site.

Light traveling along the first light path 306 passes from the first optical component 102 of the surgical contact lens 100 to a first optical element 310 within the relay optics module 304. The light then continues to mirror 315 where it is directed laterally to mirror 314. Mirror 314 then directs the light into the first optical system 318. The light then passes through the first set 322 of optical elements 325 into the eyepiece 321 where it may be perceived by a user. In this manner, the first optical component 102 acts as a first objective lens system for the first optical system 318 of the microscope system 300.

Similarly, light traveling along the second light path 308 passes from the second optical component 106 of the surgical contact lens 100 to a second optical element 312 within the relay optics module 304. The light then continues to mirror 317 where it is directed laterally to mirror 316. Mirror 316 then directs the light into the second optical system 320. The light then passes through the second set 324 of optical elements 327 into the eyepiece 323 where it may be perceived by the user. In this manner, the second optical component 106 acts as a second objective lens system for the second optical system 320 of the microscope system 300.

As a human viewer observes a surgical site, such as the ILM 202 and the retina 204 through the microscope, the viewer's left eye is presented with the first view 116 from the first optical component 102 of the surgical contact lens 100. Additionally, the viewer's right eye is presented with the second view 118 from the second optical component 106 of the surgical contact lens 100. Because the viewer's eyes are provided with two separate views 116, 118 from two distinct lateral positions, there is a greater angular disparity between the two views 116, 118. Thus, the user is provided with better depth perception of the ILM 202 and the retina 204.

In some examples, the relay optics module 304 may include additional elements such as beam-splitters (not shown). Such beam-splitters may be used, for example, in combination with illumination systems that direct light at the surgical site. Additionally, such beam-splitters may be used to direct light to image capturing devices such as a Charge Coupled Device (CCD) or Complementary Metal-Oxide Semiconductor (CMOS) imaging system.

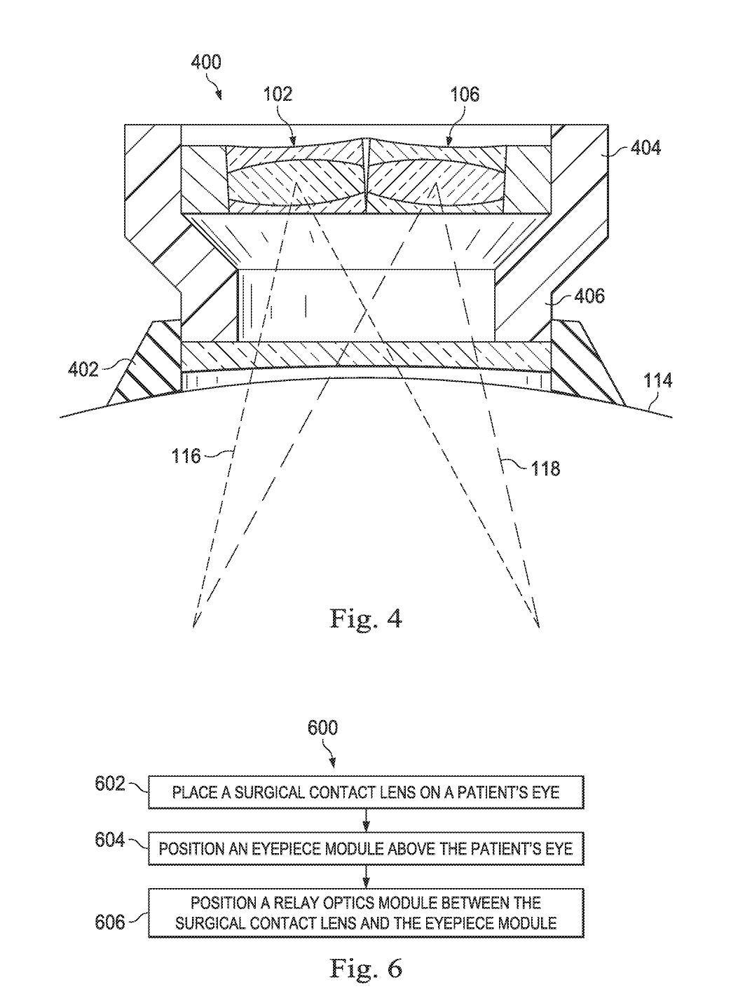

FIG. 4 is a diagram showing an illustrative cross-sectional view of a surgical contact lens 400 placed within an eye ring 402 that is secured to the patient's eye 114. According to the present example, the surgical contact lens 400 includes an upper portion 404 and a lower portion 406. The upper portion 404 may have a larger width than the lower portion 406. For example, in the case that the surgical contact lens 400 is substantially circular from the top perspective, the upper portion 404 may have a larger diameter than the lower portion 406. The upper portion 404 may be larger in order to hold the optical components 102, 106 in place. The lower portion 406 may be smaller in order to fit within an eye ring 402.

The eye ring 402 may be secured to the patient's eye 114 so that when the surgical contact lens 400 is inserted within the eye ring 402, the surgical contact lens 400 remains in place. The eye ring 402 may be secured to the patient's eye 114 in a variety of manners. In one example, the eye ring 402 is sutured to the cornea so that it may be held in place. In some examples, a transparent gel may be placed between the patient's eye 114 and the surgical contact lens 400. Such a transparent gel may remove any air bubbles between the patient's eye and the surgical contact lens 400.

In some examples, the surgical contact lens 400 is easily removable from the eye ring 402 so that the surgical contact lens 400 can be easily replaced during surgical operations. For example, there may be a set of surgical contact lenses similar to surgical contact lens 400 but differing slightly in the type of lenses used in the optical components 102, 106. These variations in the optical components 102, 106 may provide the user with different views or different perspectives of the surgical site. For example, one surgical contact lens may be designed to provide a wider angle view of the surgical site. Other surgical contact lenses may be designed to provide a more focused view of the surgical site.

FIGS. 5A and 5B are diagrams showing top views of surgical contact lenses that are arranged to provide a stereo-optic view patient's eye. FIG. 5A illustrates a surgical contact lens 500 and which the optical components 102, 106 are adjacent and abutted to each other. The surgical contact lens 500 includes a support structure 502 that holds the optical components 102, 106. In some examples, the optical components 102, 106 and the support structure 502 form a single monolithic, integral structure.

FIG. 5B illustrates a surgical contact lens 510 in which the optical components 102, 106 are separated from each other by a small distance 508. The surgical contact lens 510 includes a support structure 512 that holds the optical components 102, 106 in place. Other configurations of the optical components 102, 106 are contemplated as well. For example, in some implementations, the optical components 102, 106 may have a variety of shapes, including square, rectangular, elliptical, or other suitable shapes.

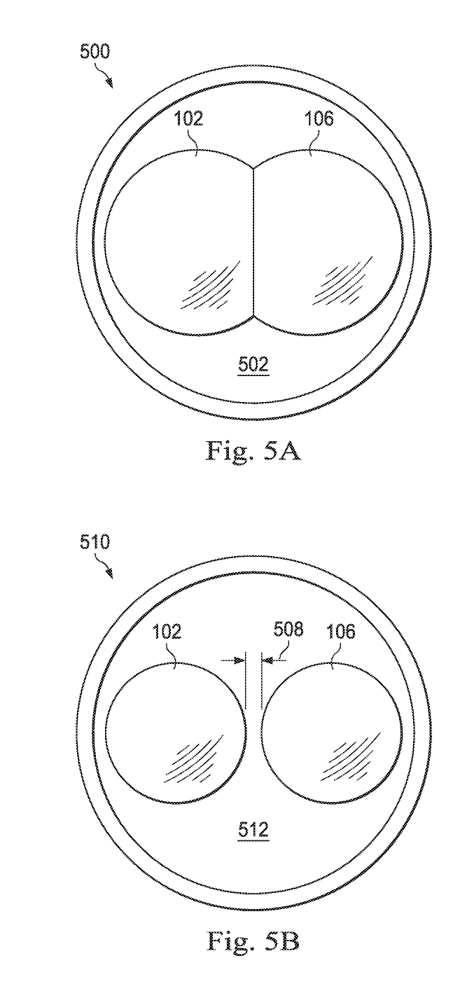

FIG. 6 is an example flowchart showing an illustrative method 600 of viewing a patient's eye through a surgical contact lens that is arranged to provide a stereo-optic view of the patient's eye. According to the present example, the method 600 includes a step 602 for placing a surgical contact lens on a patient's eye. As described above, in some examples this step includes first securing an eye ring to the patient's eye before putting the surgical contact lens in place. The eye ring may be secured to the patient's eye in a variety of manners, including friction mechanisms, suturing, or others. After the eye ring is secured, the surgical contact lens is placed in direct contact with the patient's eye. In some examples, a transparent gel is placed either on the patient's eye or on the surface of the surgical contact lens before the surgical contact lens is put in place.

According to the present example, the method 600 further includes a step 604 for positioning an eyepiece module above the patient's eye. As described above, the eyepiece module includes a first optical system for a user's left eye and a second optical system for the user's right eye. In some examples, the distal end of the eyepiece module may be placed within a range of about 3 inches to 10 inches from the surgical contact lens. Other ranges are contemplated as well.

According to the present example, the method 600 further includes a step 606 for positioning a relay optics module between the surgical contact lens and the eyepiece module. In some examples, the relay optics module is secured to the distal end of the eyepiece module. In such example, the eyepiece module and the relay optics module are positioned such that the relay optics module is directed at the surgical contact lens in the appropriate manner. Specifically the relay optics module is positioned such that light from the first optical component of the surgical contact lens is directed to the first optical system of the eyepiece module and light from the second optical component of the surgical contact lens is directed to the second optical system of the eyepiece module.

After the eyepiece module and the relay optics module are appropriately positioned, a user may view the surgical site through the microscope system. Because the surgical contact lens includes two separate optical components positioned at two lateral positions that are relatively close to the surgical site, the user is provided with better depth perception within the patient's eye. This may make it easier for the user to view the ILM, which as described above, is very thin (3 microns thick), transparent, and featureless. By providing the user with a better view of the surgical site, surgical procedures may be performed or efficiently and with better patient outcomes.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A surgical contact lens for providing a stereo-optic view inside a patient's eye, the surgical contact lens comprising:
a first side adapted to engage the patient's eye; and
a second side facing away from the patient's eye, the second side comprising:
a first optical component arranged to focus light from a first position; and
a second optical component arranged to focus light from a second position that is different from the first position,
wherein the first optical component is substantially coplanar with the second optical component.

2. The surgical contact lens of claim 1, wherein the first optical component comprises a first set of lenses arranged to act as an objective lens for a user's right eye.

3. The surgical contact lens of claim 2, wherein the second optical component comprises a second set of lenses arranged to act as an objective lens for the user's left eye.

4. The surgical contact lens of claim 1, wherein the first optical component is adjacent the second optical component.

5. The surgical contact lens of claim 1, wherein the first optical component is in direct contact with the second optical component.

6. The surgical contact lens of claim 1, wherein the first optical component in the second optical component comprise at least one of: a biconcave lens, a biconvex lens, a convex-concave lens, a plano concave lens, a plano convex lens, a positive/negative meniscus lens, an aspheric lens, a converging lens, a diverging lens, and a prism lens.

7. The surgical contact lens of claim 1, wherein the first side is smaller in diameter than the second side.

8. The surgical contact lens of claim 1, wherein the surgical contact lens is substantially circular from a top-view.

9. A microscope system for providing a stereo-optic view of a patient's eye, the system comprising:
a surgical contact lens comprising:
a first side adapted to engage a patient's eye; and
a second side facing away from the patient's eye when the first side is engaged with the patient's eye, the second side comprising a first optical component and a second optical component;
eyepiece module comprising:
a left eyepiece; and
a right eyepiece; and
a relay optics module comprising:
a first set of optical elements to direct light along a first light path from the first optical component to the left eyepiece; and
a second set of optical elements to direct light along a second light path from the second optical component to the right eyepiece.

10. The microscope system of claim 9, wherein the first light path does not overlap the second light path.

11. The microscope system of claim 9, wherein the first optical component is coplanar with the second optical component.

12. The microscope system of claim 9, wherein the first optical component is adjacent the second optical component.

13. The microscope system of claim 9, wherein the first optical component comprises a series of lenses that form an objective for the left eyepiece.

14. The microscope system of claim 9, wherein the second optical component comprises a series of lenses that form an objective for the right eyepiece.

15. The microscope system of claim 9, wherein the first set of optical elements and the second set of optical elements comprise at least one of: mirrors, beam-splitters, and lenses.

16. The microscope system of claim 9, wherein the left eyepiece and the right eyepiece each comprise a series of lenses.

17. The microscope system of claim 9, wherein the surgical contact lens is adapted to fit into an eye ring secured to the eye.

18. A method for viewing a patient's eye with a microscope, the method comprising:
placing a surgical contact lens on a patient's eye, surgical contact lens comprising a first optical component and a second optical component, the first optical component and the second optical component being substantially coplanar;
positioning an eyepiece module above the patient's eye, the eyepiece module comprising a left eyepiece and a right eyepiece; and
positioning a relay optics module between the surgical contact lens and the eyepiece module, the relay optics module comprising a first set of optical elements to direct light along a first light path from the first optical component to the left eyepiece and a second set of optical elements to drive light along a second light path from the second optical component to the right eyepiece.

19. The method of claim 18, wherein the first optical component is adjacent the second optical component.

20. The method of claim 18, wherein the first optical component and the second optical component are positioned to provide views of a surgical site from two different positions.
